(19) United States
(12) Patent Application Publication
(10) Pub. No.: US 2024/0180597 A1
(43) Pub. Date: Jun. 6, 2024

(54) BONE TIE METHODS

(71) Applicants: Jason Blain, Encinitas, CA (US); Peter Newton, San Diego, CA (US)

(72) Inventors: Jason Blain, Encinitas, CA (US); Peter Newton, San Diego, CA (US)

(21) Appl. No.: 18/529,490

(22) Filed: Dec. 5, 2023

Related U.S. Application Data
(60) Provisional application No. 63/386,232, filed on Dec. 6, 2022.

(51) Int. Cl. A61B 17/70; A61B 17/00; A61B 17/56

(52) U.S. Cl. CPC A61B 17/7071; A61B 17/7053; A61B 2017/00004; A61B 2017/564

(57) ABSTRACT

Various embodiments of bone ties and methods for treating the spine are provided. The method can include forming a first lumen in a pedicle on a first side of a pars fracture. The method can include forming a second lumen in a lamina on a second side of the pars fracture. The method can include positioning a bone tie through the first lumen and the second lumen, the bone tie comprising a distal end and a fastener section. The method can include tightening the bone tie by passing the distal end of the bone tie through the fastener section of the bone tie.

### CROSS-REFERENCE TO RELATED APPLICATIONS

This application claims priority benefit to U.S. Provisional Patent Application No. 63/386,232, filed Dec. 6, 2022, the entirety of which is hereby incorporated by reference herein.

### FIELD

Some embodiments described herein relate generally to systems and methods for performing spinal fusion and, in particular, to methods for treating a pars fracture or spondylolysis of a vertebral body.

### DESCRIPTION OF THE RELATED ART

Traumatic, inflammatory, and degenerative disorders of the spine can lead to severe pain and loss of mobility. According to some studies, back and spinal musculoskeletal impairments are the leading causes of lost work productivity in the United States. Pain as a result of some type of spinal impairment may have its source in a variety of pathologies or clinical conditions.

One source for back and spine pain is related to a pars fracture or spondylolysis. This is a stress fracture through the pars interarticularis of the vertebra, typically the lumbar vertebra. Pars fractures can cause spondylolisthesis, which is the sliding forward of one vertebral body over the adjacent vertebral body. While many technological advances have focused on the spinal disc and artificial replacement or repair of the disc, little advancement in pars fracture repair has been made. Thus, there is a need to address the clinical concerns raised by pars fracture.

The current standard of care to address pars fracture is activity modification to allow rest to promote healing, bracing of the lumbar spine, steroid injections, and/or physical therapy. However, for some patients, pain is persistent and surgical intervention is needed. One option is to fuse the two adjacent bone portions together by placing a screw through the pars to bridge the fracture. This surgical procedure can lead to further complications and unsuccessful fusion where a large gap in the pars remains.

### SUMMARY

Devices and methods are disclosed for treating the vertebral column. In some embodiments, a bone tie for securing or fusing a pars fracture is provided. The bone tie can be used to stabilize and/or fixate a first bone portion to a second bone portion. The bone tie can be used to reduce the pain. The bone tie can be used to reduce further degradation of a spine. The bone tie can be used to stabilize or fixate the pars interarticularis after a break. The bone tie can be used to form a loop around a pars fracture.

In some embodiments, a method of treating a patient is provided. The method can include forming a first lumen in a pedicle on a first side of a pars fracture. The method can include forming a second lumen in a lamina on a second side of the pars fracture. The method can include positioning a bone tie through the first lumen and the second lumen, the bone tie comprising a distal end and a fastener section. The method can include tightening the bone tie by passing the distal end of the bone tie through the fastener section of the bone tie.

In some embodiments, forming the first lumen comprises drilling a hole entirely through the pedicle. In some embodiments, forming the first lumen comprises drilling a hole partially through the pedicle. In some embodiments, forming the second lumen comprises drilling a hole entirely through the lamina. In some embodiments, forming the second lumen comprises drilling a hole partially through the lamina. In some embodiments, forming the second lumen comprises connecting the second lumen and the first lumen. In some embodiments, positioning the bone tie comprises wrapping the bone tie around the outside portion of the vertebra. In some embodiments, tightening the bone tie further comprises reducing a unilateral pars fracture. In some embodiments, tightening the bone tie further comprises applying compression to the pars fracture. In some embodiments, tightening the bone tie further comprises uniting fractured ends of the pars interarticularis. In some embodiments, the method can include removing the distal end of the bone tie. In some embodiments, the method can include removing the bone tie after the pars fracture heals. In some embodiments, the bone tie comprises a bioabsorbable or bioresorbable material.

In some embodiments, a method of treating a patient is provided. The method can include forming a first lumen in a first pedicle on a first side of a first pars fracture. The method can include forming a second lumen in a first lamina on a second side of the first pars fracture. The method can include positioning a first bone tie through the first lumen and the second lumen, the first bone tie comprising a distal end and a fastener section. The method can include tightening the first bone tie by passing the distal end of the first bone tie through the fastener section of the first bone tie. The method can include forming a third lumen in a second pedicle on a first side of a second pars fracture. The method can include forming a fourth lumen in a second lamina on a second side of the second pars fracture. The method can include positioning a second bone tie through the third lumen and the fourth lumen, the second bone tie comprising a distal end and a fastener section. The method can include tightening the second bone tie by passing the distal end of the second bone tie through the fastener section of the second bone tie.

In some embodiments, forming the second lumen comprises connecting the second lumen and the first lumen. In some embodiments, forming the fourth lumen comprises connecting the fourth lumen and the third lumen. In some embodiments, tightening the first bone tie and tightening the second bone tie further comprises reducing a bilateral pars fracture. In some embodiments, tightening the first bone tie further comprises applying compression to the first pars fracture and tightening the second bone tie comprises applying compression to the second pars fracture. In some embodiments, tightening the first bone tie and tightening the second bone tie further comprises correcting a slip of a vertebra. In some embodiments, tightening the first bone tie and tightening the second bone tie further comprises correcting a displaced fracture.

### BRIEF DESCRIPTION OF THE DRAWINGS

The structure and method of use will be better understood with the following detailed description of embodiments, along with the accompanying illustrations, in which:

FIG. 1 is a lateral view of a portion of the vertebral column.

FIG. 2 is a perspective front view of an embodiment of a bone tie.

FIG. 3 is a perspective back view of the bone tie of FIG. 2.

FIG. 4 is a perspective view of a proximal portion of the bone tie of FIG. 2.

FIG. 5 is a perspective view of a distal portion of the bone tie of FIG. 2.

FIG. 6 is an enlarged perspective view of a distal portion of the bone tie of FIG. 2.

FIG. 7 is a perspective view of an embodiment of a bone tie.

FIG. 8 is a perspective view of the bone tie of FIG. 7.

FIG. 9 is a perspective view of an embodiment of a bone tie.

FIG. 10 is a side cross-sectional view of the bone tie of FIG. 9.

FIG. 11 is a top view of the bone tie of FIG. 9.

FIG. 12 is a flow chart for a method of using the bone tie.

FIGS. 13-14 are views of a method of repairing a pars fracture.

FIG. 15 is a flow chart for a method of using bone ties.

FIGS. 16-17 are views of a method of repairing pars fractures.

### DETAILED DESCRIPTION

Although certain preferred embodiments and examples are disclosed below, it will be understood by those in the art that the disclosure extends beyond the specifically disclosed embodiments and/or uses of the invention and obvious modifications and equivalents thereof. Thus, it is intended that the scope should not be limited by the particular disclosed embodiments described below.

The systems and methods described herein relate to embodiments of bone ties and methods of use. The methods can include treating a pars fracture of a vertebral body. The methods can include drilling a hole in a pedicle of the vertebral body. The methods can include drilling a hole at least partially through the lamina, connecting to the hole in the pedicle. The methods can include passing a bone tie through the holes and wrapping the bone tie back around the outside, dorsal portion of the vertebral body. The methods can include tensioning and securing the bone tie to reduce the fracture.

### 1. Anatomy of the Spine

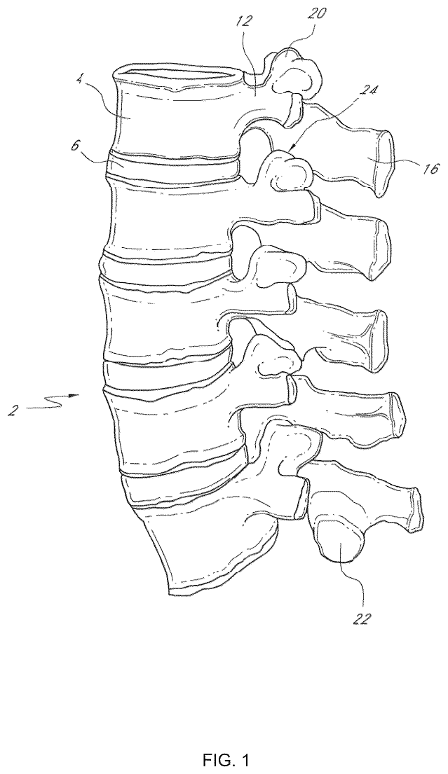

As shown in FIG. 1, the vertebral column 2 comprises a series of alternating vertebrae 4 and fibrous discs 6 that provide axial support and movement to the upper portions of the body. The vertebral column 2 typically comprises thirty-three vertebrae 4, with seven cervical (C1-C7), twelve thoracic (T1-T12), five lumbar (L1-15), five fused sacral (S1-S5) and four fused coccygeal vertebrae. Each thoracic vertebra includes an anterior body with a posterior arch. The posterior arch comprises two pedicles 12 and two laminae that join posteriorly to form a spinous process 16. Projecting from each side of the posterior arch is a transverse, superior 20 and inferior articular process 22. The facets 24 of the superior 20 and inferior articular processes 22 form facet joints with the articular processes of the adjacent vertebrae. The facet joints are true synovial joints with cartilaginous surfaces and a joint capsule. The facet joints guide the motion of the spine in certain directions. The facet joints also prevent the superior vertebra from slipping forward relative to the inferior vertebra.

The pars interarticularis connects the facet joints. In particular, the facet joints above are connected to the facet joints below. Fractures of the pars interarticularis can be called a pars defect or a pars fracture. A pars fracture is typically a stress fracture. This type of fracture typically occurs in the lumbar vertebra. The fracture may occur only on one side of the vertebra, or may occur on both sides of the vertebra. When both sides of the vertebra are fractured, spondylolisthesis may occur wherein one vertebra slides relative to another vertebra.

Pars fractures can be resistant to healing. The surface area of the fracture is small, leading to less healing potential. The pars interarticularis lacks substantial cancellous bone, leading to less healing potential. The area of the fracture is located in an area subjected to motion, leading to less healing potential. The ends of the fracture may be misaligned due to slippage, leading to less healing potential. Persistent symptoms including pain may necessitate surgical intervention.

### 2. Bone Tie

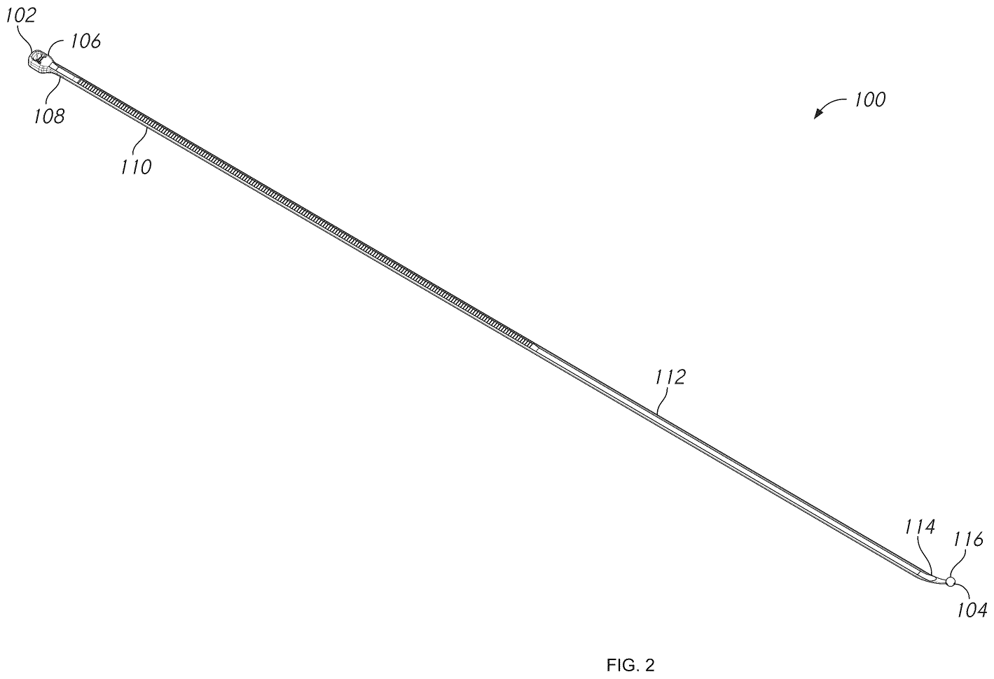

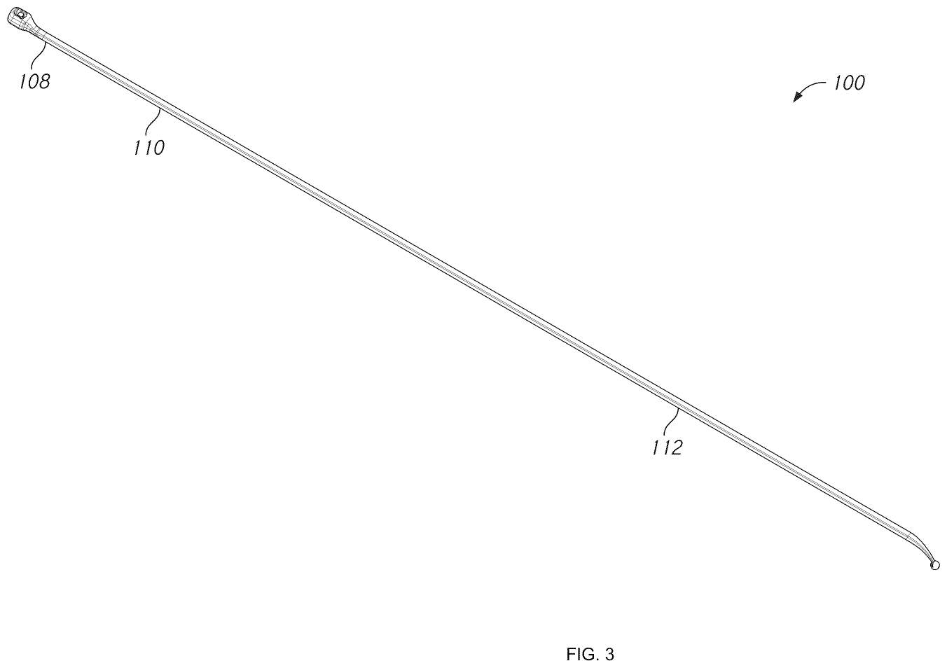

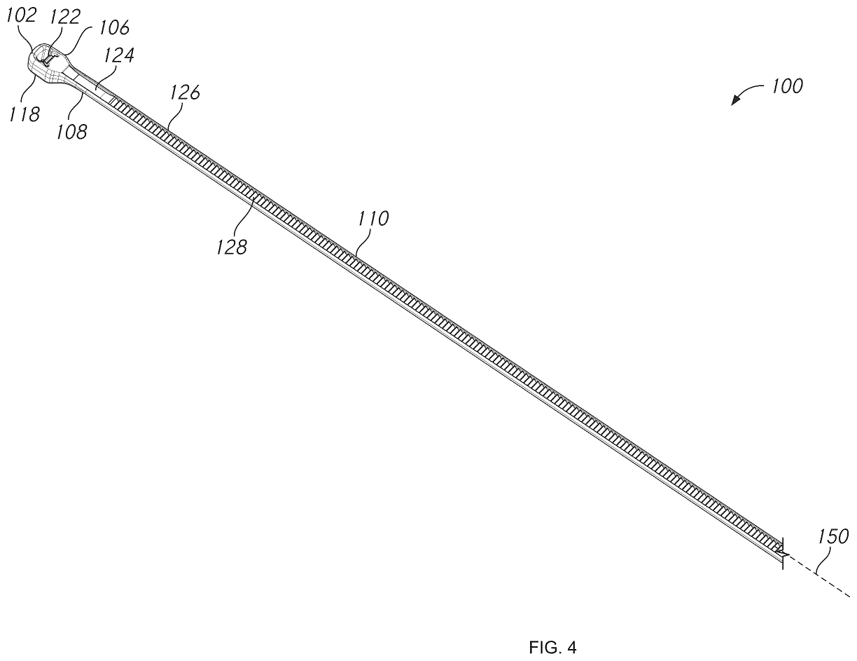

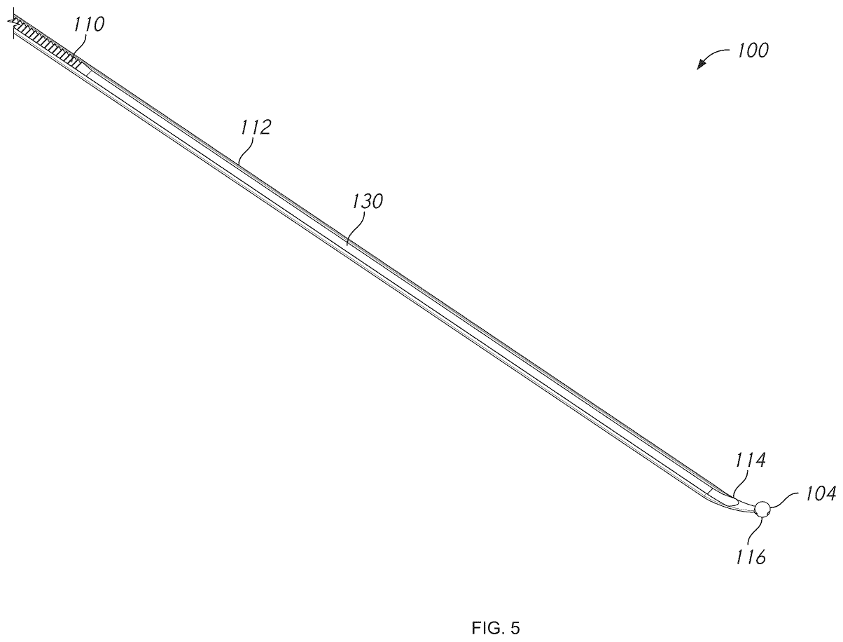

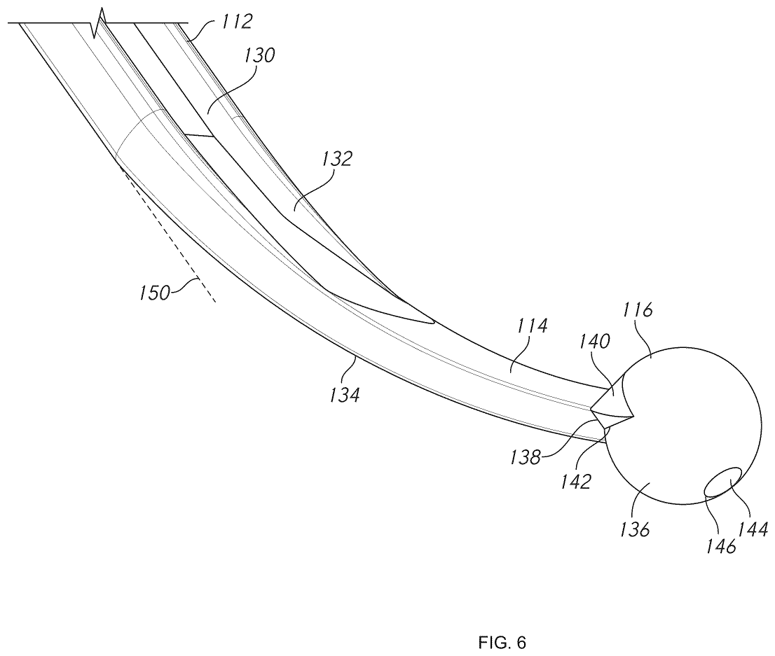

FIGS. 2-11 depict views of embodiments of bone ties. FIG. 2 illustrates a perspective front view of a bone tie 100. FIG. 3 illustrates a perspective back view of the bone tie 100. FIG. 4 illustrates a perspective view of a proximal portion of the bone tie 100. FIG. 5 illustrates a perspective view of a distal portion of the bone tie 100. FIG. 6 illustrates an enlarged perspective view of a distal portion of the bone tie 100.

FIG. 2 is a perspective front view of the bone tie 100. The bone tie 100 can be a generally elongate member. The bone tie 100 can comprise a proximal end 102 and a distal end 104. The bone tie 100 can include a length between the proximal end 102 and the distal end 104. The proximal end 102 can be configured to be near the hands of the user when the user is manipulating the bone tie 100 or using a bone tie inserter. The distal end 104 can be configured to be inserted into a bone lumen as described herein. The distal end 104 can be configured to be the first portion of the bone tie 100 that is inserted in the lumen. The distal end 104 can be the leading end of the bone tie 100. In some embodiments, the proximal end 102 extends away from the vertebra during insertion of the bone tie 100. In some embodiments, the proximal end 102 is held by the user. In some embodiments, the proximal end 102 is unconstrained during insertion of the bone tie 100. In some embodiments, bone tie 100 can be grasped and manipulated by a user.

The bone tie 100 can include one or more sections along the length of the bone tie 100. The sections can have a different shape, configuration, or function than an adjacent section of the bone tie 100. In some embodiments, one or more non-adjacent sections can have the same shape, configuration, or function as another section of the bone tie 100. In some embodiments, one or more additional sections are provided. In some embodiments, one or more of the sections provided herein are omitted.

The bone tie 100 can include a fastener section 106. The fastener section 106 can be located at or near the proximal end 102. The fastener section 106 can include any mechanism configured to secure the fastener section 106 to another section of the bone tie 100. The fastener section 106 can include a mechanism that allows the bone tie 100 to be secured in a single direction of travel such as a ratchet. The fastener section 106 can include a mechanism that allows the bone tie 100 to be secured in two directions of travel such as a pair of gears.

The bone tie 100 can include a first section 108. The first section 108 can be closer to the proximal end 102 than the distal end 104. The first section 108 can have a first cross-sectional shape. The first section 108 can extend distally from the fastener section 106. The bone tie 100 can include a second section 110. The second section 110 can be closer to the proximal end 102 than the distal end 104. The second section 110 can have a second cross-sectional shape. The second section 110 can extend distally from the first section 108. The bone tie 100 can include a third section 112. The third section 112 can be closer to the distal end 104 than the proximal end 102. The third section 112 can have a third cross-sectional shape. The third section 112 can extend distally from the second section 110.

The bone tie 100 can include a neck section 114. The neck section 114 can be closer to the distal end 104 than the proximal end 102. The neck section 114 can taper from the third section 112 toward the distal end 104. The neck section 114 can extend distally from the third section 112. The neck section 114 can facilitate manipulation of the distal portion of the bone tie 100 by the bone tie inserter. The neck section 114 can be shaped to interface with the bone tie inserter. The neck section 114 can be shaped to form a mechanical interfit or coupling.

The bone tie 100 can include a head section 116. The head section 116 can be located at or near the distal end 104. The neck section 114 can taper toward the head section 116. The head section 116 can extend distally from the neck section 114. The head section 116 can facilitate manipulation of the distal portion of the bone tie 100 by the bone tie inserter. The head section 116 can be shaped to be grasped or cupped by the bone tie inserter. The head section 116 can be shaped to pivot and/or rotate relative to the bone tie inserter.

FIG. 3 is a perspective back view of the bone tie 100. The bone tie 100 can have a smooth surface along the first section 108, the second section 110, and the third section 112. The bone tie 100 can have a continuous surface along the first section 108, the second section 110, and the third section 112.

FIG. 4 illustrates a perspective view of a proximal portion of the bone tie 100. The bone tie can include the proximal end 102, the fastener section 106, first section 108, and the second section 110.

The fastener section 106 can include a lumen 118. The lumen 118 can be oriented perpendicular to a longitudinal axis 150 of the bone tie 100. The bone tie 100 can include a ratchet 122 disposed within the lumen 118. The ratchet 122 is configured to deflect to allow one or more gears to travel through the lumen 118 in one direction, but limit or prevent travel in another direction. The fastener section 106 can form an enlarged end of the bone tie 100. The fastener section 106 can be generally rectangular or cuboid. The fastener section 106 can have a width larger than the first section 108. The fastener section 106 can have a thickness larger than the first section 108. The fastener section 106 can include rounded edges or corners. The fastener section 106 can have any shape to accommodate the ratchet 122 disposed therewithin. The fastener section 106 can have any shape to accommodate any fastener mechanism described herein.

The first section 108 can have the first cross-sectional shape. The first cross-sectional shape can be generally rectangular or cuboid. The first cross-sectional shape can have rounded edges or corners. The first section 108 can include a width and a thickness. The first section 108 can include a groove 124. The groove 124 can reduce the thickness of the first section 108. The groove 124 can taper from the fastener section 106. The groove 124 can taper to the second section 110.

The second section 110 can have the second cross-sectional shape. The second cross-sectional shape can be generally rectangular or cuboid. The second cross-sectional shape can have rounded edges or corners. The second section 110 can include a groove 126. The groove 124 of the first section 108 can extend to the groove 126 of the second section 110. The second section 110 can include one or more gears 128. The gears 128 can be ramped surfaces. The gears 128 can form a rack. The gears 128 can be wedge surfaces. The gears 128 can be inclined upward toward the proximal end 102. The gears 128 can be inclined downward toward the distal end 104. The gears 128 can be disposed within the groove 126 of the second section 110. The first section 108 and the second section 110 can include a constant width. The first section 108 and the second section 110 can include a constant thickness. The first section 108 and the second section 110 can include a constant thickness measured along the edges of the first section 108 and the second section 110.

FIG. 5 illustrates a perspective view of a distal portion of the bone tie 100. The bone tie can include the second section 110, the third section 112, the neck section 114, the head section 116, and the distal end 104.

The third section 112 can have a third cross-sectional shape. The third cross-sectional shape can be generally rectangular or cuboid. The third cross-sectional shape can have rounded edges or corners. In some embodiments, the first cross-sectional shape and the third cross-sectional shape are the same or similar. The third section 112 can include a width and a thickness. The third section 112 can include a groove 130. The groove 130 can reduce the thickness of the third section 112. The groove 130 can taper from the second section 110. The groove 130 can taper to the neck section 114.

Two or more of the first section 108, the second section 110, and the third section 112 can include a constant width. Two or more of the first section 108, the second section 110, and the third section 112 can include a constant thickness. Two or more of the first section 108, the second section 110, and the third section 112 can include a constant thickness measured along the edges of the respective sections. The bone tie 100 can have a constant width along a substantial portion of the length. The bone tie 100 can have a constant thickness along a substantial portion of the length.

FIG. 6 illustrates an enlarged view of the distal portion of the bone tie 100. The bone tie 100 can include the neck section 114. The neck section 114 tapers along the width. The neck section 114 tapers from a larger width near the third section 112 to a smaller width near the head section 116. The neck section 114 can include a groove 132. The groove 132 can reduce the thickness of the neck section 114. The groove 132 of the neck section 114 can extend from the groove 130 of the third section 112.

The neck section 114 can lie in a plane along the longitudinal axis 150 of the bone tie 100 or the neck section 114 can include a curve 134. The curve 134 can have a constant radius of curvature. Two or more of the first section 108, the second section 110, and the third section 112 can be planar. The bone tie 100 can lie in a plane along a substantial portion of the length. The curve 134 can extend from the plane of the bone tie. The curve 134 can extend upward from the grooves 124, 126, 130, 132 of the bone tie 100. The curve 134 can extend upward from the gears 128 of the second section 110. The curve 134 can extend away from the longitudinal axis 150 of the bone tie 100.

The bone tie 100 can include the head section 116. The head section 116 can include a head 136. The head 136 can be rounded. The head 136 can be spherical. The head 136 can extend to the distal end 104 of the bone tie 100. The head section 116 can include a flange 138. The flange 138 can be positioned on the head 136. The flange 138 can be a rounded bill that extends from the head 136. The flange 138 can include a first tapered surface 140 and a second tapered surface 142. The first tapered surface 140 and the second tapered surface 142 can have different slopes. The second tapered surface 142 can form a ledge by which the head section 116 or head 136 can be grasped. The first tapered surface 140 and the second tapered surface 142 extend to the neck section 114.

The bone tie 100 can include a marker 144. The marker 144 can facilitate visualization of the bone tie 100, or a portion thereof. In the illustrated embodiment, the head 136 can include the marker 144. The head 136 can include a bore 146. The bore 146 can extend from an edge of the head 136 inward toward or past the center of the head 136. The marker 144 can be disposed within the bore 146. The marker 144 can be a radiopaque marker. The marker 144 can be formed of a metal or other radiopaque material. The marker 144 can identify the distal end 104 of the bone tie 100. In some embodiments, the bone tie 100 comprises a non-radiopaque material. In some embodiments, one or more radiopaque markers may be embedded in or on the bone tie 100 to assist in placement or monitoring of the bone tie 100 under radiographic visualization.

The bone tie 100 can be a flexible fastening band. The bone tie 100 can include the proximal end portion 102 and the distal end portion 104. In some embodiments, the head section 116 can be removed. The neck section 114 can be advanced through the lumen 118. When the neck section 114 is advanced, the ratchet 122 can extend into the groove 132. The third section 112 can be advanced through the lumen 118. When the third section 112 is advanced, the ratchet 122 can extend into the groove 130. The second section 110 can be advanced through the lumen 118. When the second section 110 is advanced, the ratchet 122 can extend into the groove 126. The ratchet 122 can engage the gears 128. The ratchet 122 can allow the second section 110 to travel through the lumen 118 in one direction, but limit travel through the lumen 118 in the opposite direction.

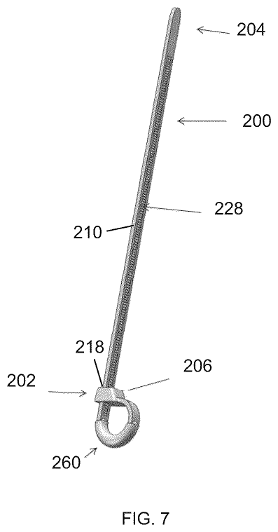

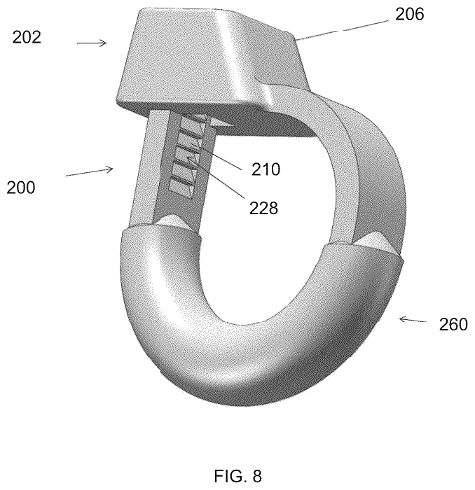

FIGS. 7 and 8 are perspective views of a bone tie 200. The bone tie 200 can include any of the features of the bone tie 100. The bone tie 200 can comprise a proximal end 202 and a distal end 204. The distal end 204 can be the leading end of the bone tie 200. The bone tie 200 can include one or more sections along the length of the bone tie 200. The sections can have a different cross-sectional shape than an adjacent section of the bone tie 200. The sections can have a different length than an adjacent section of the bone tie 200. The sections can have a different function than an adjacent section of the bone tie 200. The bone tie 200 can include a fastener section 206. The fastener section 206 can be located at or near the proximal end 202. The fastener section 206 can include a mechanism configured to form a loop. The fastener section 206 can include a mechanism that allows the bone tie 200 to be tightened in a single direction.

The distal end 204 of the bone tie 200 can have a tapered tip. The distal end 204 can facilitate passage through the fastener section 206. The fastener section 206 can include a lumen 218. The lumen 218 can be oriented through the fastener section. The bone tie 100 can include a ratchet disposed within the lumen 218. The ratchet is configured to engage one or more gears. The bone tie 200 can include at least a portion that includes one or more gears. The bone tie 200 can include at least a portion that does not include one or more gears. The bone tie 200 can have any cross-sectional shape. The section 210 can include one or more gears 228. The gears 228 can be ramped surfaces. The gears 228 can form a rack. The gears 228 can be wedge surfaces. The gears 228 can be inclined toward the proximal end 202 or the distal end 204. The gears 228 can be declined toward the proximal end 202 or the distal end 204. The fastener section 206 can include a ratchet 222 configured to engage the gears 228 formed in the bone tie 200. The fastener section 206 can allow the gears 228 to advance through the lumen 218 in only one direction.

The distal end 204 is configured to pass through a lumen formed through a vertebra. The distal end 204 is configured to pass through a lumen in an interbody device. The distal end 204 is configured to pass through the lumen 218 in the fastener section 206. The distal end 204 can be shaped to increase the ease of inserting the distal end 204 into the lumen 218 in the fastener section 206. The distal end 204 can be tapered, rounded, and/or angled to reduce at least a portion of a cross-sectional area of the distal end 204. The bone tie 200 can be monolithically formed.

The bone tie 200 can include one or more additional portions 260. The bone tie 200 can include a cylindrical portion. The bone tie 200 can include a cuboid portion. The bone tie 200 can include a portion having substantially the same cross-sectional dimension as the cross-sectional dimension of the lumen of a vertebra. The bone tie 200 can include a portion having substantially the same cross-sectional dimension as the cross-sectional dimension of a notch in an interbody device.

The bone tie 200 can have an excess length. The excess length can be removed, e.g., by cutting or breaking. The distal end 204 and a portion of the bone tie 200 is removed in FIG. 8. The bone tie 200 forms a loop. The bone tie 200 can be further tightened, but not loosened. The bone tie 200 can be removed by cutting or breaking the loop.

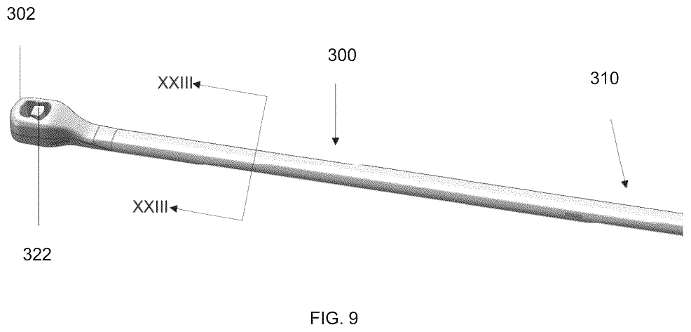

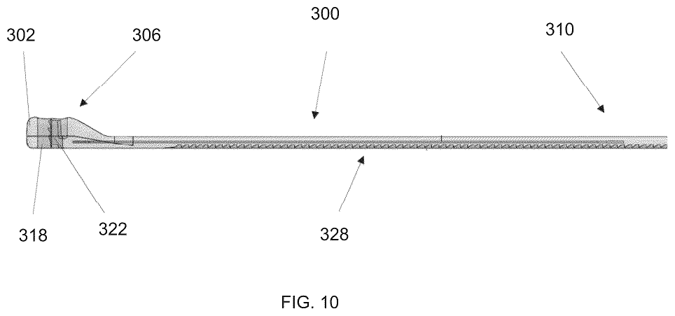

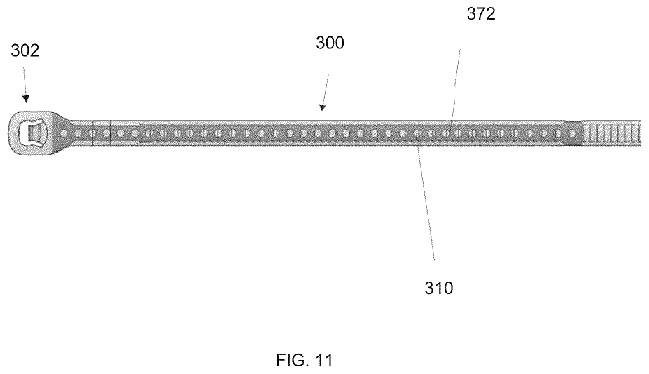

FIGS. 9-11 are views of a bone tie 300. FIG. 9 is a perspective view of the proximal portion of the bone tie 300. FIG. 10 is a side cross-sectional view of the proximal portion of the bone tie 300. FIG. 11 is a top view of the proximal portion of the bone tie 300.

The bone tie 300 can include any of the features of the bone tie 100 or bone tie 200. The bone tie 300 can comprise a proximal end 302 and a distal end (not shown). The distal end can have any features of the distal end 104, 204. The distal end can be the leading end of the bone tie 300. The bone tie 300 can include a fastener section 306. The fastener section 306 can include a mechanism that allows the bone tie 300 to be secured.

The fastener section 306 can include a lumen 318. The bone tie 300 can include a ratchet 322 disposed within the lumen 318. The ratchet 322 is configured to engage one or more gears 328 to allow tightening and securing of the bone tie 300. The bone tie 300 can include at least a portion that includes one or more gears 328. The section 310 can include one or more gears 328. The fastener section 306 can include a ratchet 322 configured to engage the gears 328. The fastener section 306 can allow the gears 328 to advance through lumen 318 in only one direction. The bone tie 300 can include a gear rack.

The bone tie 300 can include a reinforcement piece 372. The reinforcement piece 372 can include any of the materials described herein. The reinforcement piece 372 can include a material stronger than another portion of the bone tie 300. The reinforcement piece 372 can include a metal. The reinforcement piece 372 can include a polymer. The reinforcement piece 372 can be disposed within the bone tie 300. The reinforcement piece 372 can be disposed along the length of the bone tie 300. The reinforcement piece 372 can be closer to the proximal end 302 than the distal end. The reinforcement piece 372 can overlap with the one or more gears 328. The reinforcement piece 372 can be molded within the bone tie 300. The reinforcement piece 372 can have any shape. The reinforcement piece 372 can have any length. The reinforcement piece 372 can change the bending characteristics of the bone tie. The reinforcement piece 372 can change the torsion characteristics of the bone tie 300.

The bone tie 100, 200, 300 can have a width of 0.5 mm, 1 mm, 1.5 mm, 2 mm, 2.5 mm, 3 mm, 3.5 mm, 4 mm, 4.5 mm, 5 mm, 5.5 mm, 6 mm, or any range of the foregoing values. The width of the bone tie 100, 200, 300 can vary along the length of the bone tie 100, 200, 300. The bone tie 100, 200, 300 can have a thickness of 0.5 mm, 1 mm, 1.5 mm, 2 mm, 2.5 mm, 3 mm, 3.5 mm, 4 mm, or any range of the foregoing values. The thickness of the bone tie 100, 200, 300 can vary along the length of the bone tie 100, 200, 300. The bone tie 100, 200, 300 can have a length of 10 mm, 20 mm, 30 mm, 40 mm, 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 mm, 300 mm, 305 mm, 310 mm, 315 mm, 320 mm, 325 mm, 330 mm, 335 mm, 340 mm, 345 mm, 350 mm, 355 mm, 360 mm, 365 mm, 370 mm, 375 mm, 380 mm, 385 mm, 390 mm, 395 mm, 400 mm, or any range of the foregoing values. For example, the bone tie 100, 200, 300 can have a length of 175 mm. In some embodiments, the second section 110, 210, 310 or the gears 128, 228, 328 can have a length of 5 mm, 10 mm, 15 mm, 20 mm, 25 mm, 30 mm, 35 mm, 40 mm, 45 mm, 50 mm, 60 mm, 65 mm, 70 mm, 75 mm, 80 mm, or any range of the foregoing values.

The bone tie 100, 200, 300 can be manufactured from any of a variety of materials known in the art, including but not limited to a polymer such as polyetheretherketone (PEEK), polyetherketoneketone (PEKK), polyethylene, fluoropolymer, hydrogel, or elastomer; a ceramic such as zirconia, alumina, or silicon nitride; a metal such as titanium, titanium alloy, cobalt chromium or stainless steel; or any combination of the materials described herein. The bone tie 100, 200, 300 can include any biocompatible material, e.g., stainless steel, titanium, PEEK, nylon, etc. In some embodiments, the bone tie 100, 200, 300 comprises at least two materials. The bone tie 100, 200, 300 can include a reinforcement piece disposed within the bone tie 100, 200, 300. By selecting a particular configuration and the one or more materials for the bone tie 100, 200, 300, the bone tie 100, 200, 300 can be designed to have the desired flexibility and resiliency.

In some embodiments, the bone tie 100, 200, 300 can form a unitary structure. The bone tie 100, 200, 300 can be integrally formed from the proximal end to the distal end. In some embodiments, the bone tie 100, 200, 300 can include one or more unitarily formed sections along the length of the bone tie 100, 200, 300. In some embodiments, the bone tie 100, 200, 300 can include one or more separately formed sections along the length of the bone tie 100, 200, 300. The bone tie 100, 200, 300 can be monolithically formed. The bone tie 100, 200, 300 can be formed of the same or similar material. The sections of the bone tie 100, 200, 300 can be formed of the same or similar construction. In some embodiments, the bone tie 100, 200, 300 is formed from an injection molding process. In some embodiments, the shape of the bone tie 100, 200, 300 can be determined based on the shape of an artificial lumen formed through a vertebra. In some embodiments, the shape of the bone tie 100, 200, 300 can be determined based on the shape of a notch or opening formed through the interbody device.

In some embodiments, the characteristic of the bone tie 100, 200, 300 can vary along the length of the bone tie 100, 200, 300. In some embodiments, the flexibility of the bone tie 100, 200, 300 varies along the length of the bone tie 100, 200, 300. In some embodiments, the torsional strength of the bone tie 100, 200, 300 varies along the length of the bone tie 100, 200, 300. In some embodiments, the resistance to deformation or elongation of the bone tie 100, 200, 300 varies along the length of the bone tie 100, 200, 300. In some embodiments, the characteristic of the bone tie 100, 200, 300 vary based, at least in part, on the shape of the various sections.

In some embodiments, the characteristic of the bone tie 100, 200, 300 vary based on the material of the various sections. In some embodiments, the characteristic of the bone tie 100, 200, 300 vary along the length based, at least in part, on a reinforcement piece. The reinforcement piece can be separately formed from or integrally formed with the bone tie 100, 200, 300. The reinforcement piece can comprise a different material or material property. The reinforcement piece can increase the strength of a section of the bone tie 100, 200, 300. The reinforcement piece can increase or decrease bending strength. The reinforcement piece can increase or decrease torsion strength. In some embodiments, the reinforcement piece is radiopaque. In some embodiments, the reinforcement piece is radiolucent.

### 3. Methods of Use

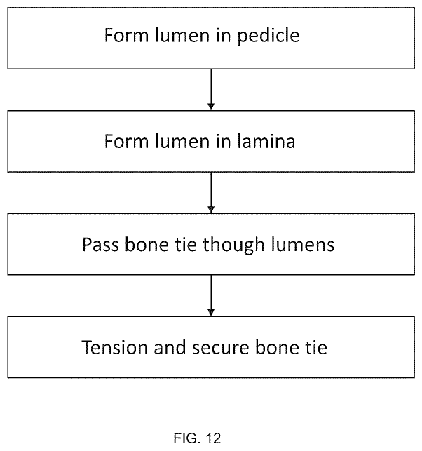

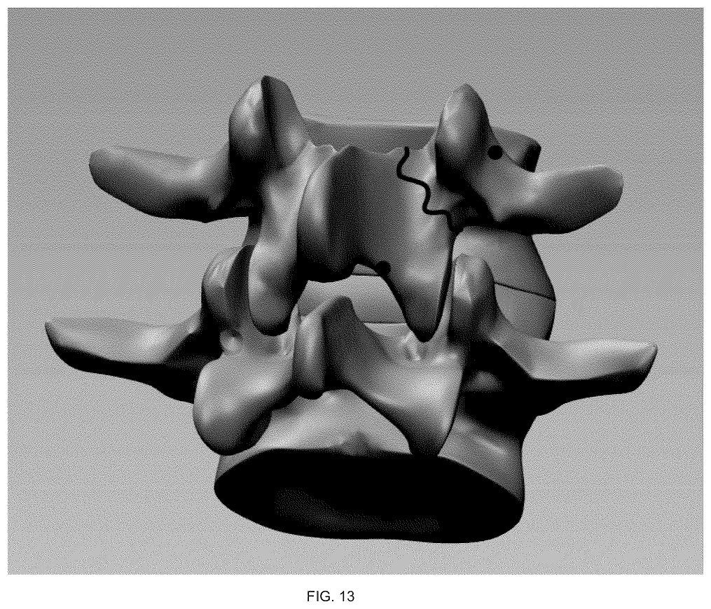

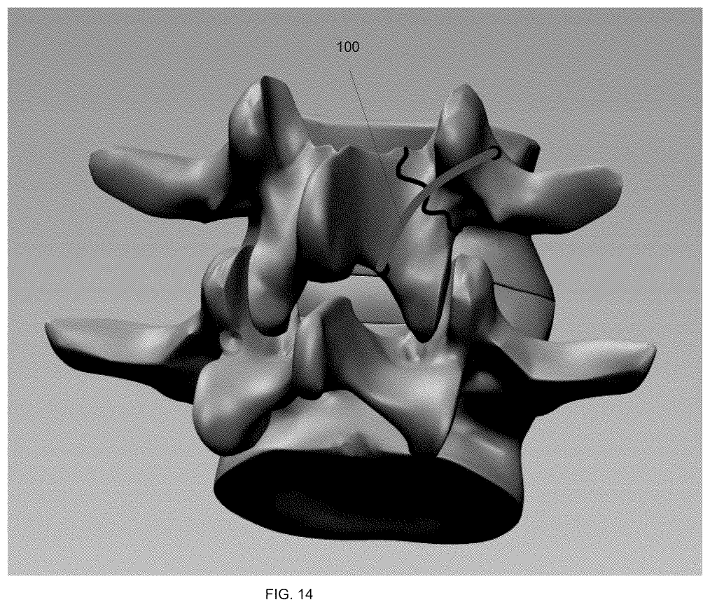

FIG. 12 is a flow chart for a method of using the bone tie. FIG. 13 illustrates a view of a vertebra with a pars fracture. FIG. 14 is a view of the bone tie 100 positioned to correct the pars fracture. The bone tie 100 can be positioned by one or more tools. The surgeon can position the bone tie 100 manually, depending on the anatomical location of fixation and the patient's anatomy. While bone tie 100 is illustrated, the methods described herein can be used with any bone tie described herein.

The bone tie 100 can be configured to correct a pars fracture. While the lumbar vertebra is shown, the fracture can occur in any vertebra. The bone tie 100 can be configured to stabilize the vertebra in a position to promote healing. The bone tie 100 can be configured to stabilize the fracture. The bone tie 100 can be configured to stabilize the bone fragments. The bone tie 100 can be configured to fuse the pars interarticularis. The bone tie 100 can be configured to fuse the bone fragments of pars interarticularis. The bone tie 100 can be anchored to one or more specific anatomical locations.

The bone tie 100 can be used to fuse the bone fragments of a single vertebra. The bone tie 100 can be used to fuse the pedicle to the lamina of a vertebra. The bone tie 100 can be used to fuse bone fragments caused by a fracture. In some embodiments, the alignment of the bone fragments is corrected by using the bone tie 100. The ends of the broken bone can be brought into alignment. The ends of the fractured bone are brought adjacent to each other. The bone tie 100 can facilitate and maintain the alignment of the ends of the fractured bone. In some embodiments, the vertebra can be treated using only one bone tie 100. In some embodiments, one bone tie 100 can be used to stabilize a fracture on the right side of the spinous process. In some embodiments, one bone tie 100 can be used to stabilize a fracture on the left side of the spinous process. In some embodiments, one bone tie 100 can be used to stabilize a fracture on the right side of the spinous process and one bone tie 100 can be used to stabilize a fracture on the left side of the spinous process. In some embodiments, the two bone ties 100 exert the same force on the bone fragments. In some embodiments, the two bone ties 100 exert different forces on the bone fragments. The methods described herein can be repeated for any fracture. For an example reference herein, the anatomical features are on the right when viewed from the front of the patient and the anatomical features are on the left when viewed from the front of the patient. Other configurations are contemplated.

In some embodiments, a pathway is formed in the pedicle. In some embodiments, a lumen is formed in the pedicle. In some embodiments, the lumen can extend entirely through the pedicle. The lumen in the pedicle can extend to the vertebral foramen. In other embodiments, the lumen can extend partially through the pedicle. The lumen in the pedicle can be formed with any tool, such as a drill or reamer. The lumen in the pedicle can be formed via a posterior approach to the spine. The lumen in the pedicle can be formed via a lateral approach to the spine. The lumen in the pedicle can be formed via any of a variety of approaches to the spine. The lumen in the pedicle can be formed via minimally invasive surgical techniques. The lumen in the pedicle is formed through the pedicle of a single vertebra. In some embodiments, at least a portion of the lumen in the pedicle has a curved or non-linear configuration. In some embodiments, at least a portion of the lumen in the pedicle has a straight or linear configuration. In some embodiments, the lumen in the pedicle has a straight or linear configuration. The lumen in the pedicle is on one side of the pars fracture. The lumen in the pedicle can be near the pars fracture. The lumen in the pedicle can be adjacent to the pars fracture. The lumen in the pedicle can be spaced apart from the pars fracture. The lumen in the pedicle can be a distance from the pars fracture of 5 mm, 10 mm, 15 mm, 20 mm, more than 5 mm, more than 10 mm, more than 15 mm, more than 20 mm, less than 5 mm, less than 10 mm, less than 15 mm, less than 20 mm, or any range of two of the foregoing values.

In some embodiments, a pathway is formed in the lamina. In some embodiments, a lumen is formed in the lamina. In some embodiments, the lumen can extend entirely through the lamina. The lumen in the lamina can extend to the vertebral foramen. In other embodiments, the lumen can extend partially through the lamina. The lumen in the lamina can be formed with any tool, such as a drill or reamer. The lumen in the pedicle and the lumen in the lamina can be formed via the same tool. The lumen in the pedicle and the lumen in the lamina can be formed via different tools. The lumen in the lamina can be formed via a posterior approach to the spine. The lumen in the lamina can be formed via a lateral approach to the spine. The lumen in the lamina can be formed via any of a variety of approaches to the spine. The lumen in the lamina can be formed via minimally invasive surgical techniques. The lumen in the pedicle and the lumen in the lamina can be formed via the same approach to the spine. The lumen in the pedicle and the lumen in the lamina can be formed via the different approaches to the spine. The lumen in the lamina is formed through the lamina of a single vertebra. The lumen in the pedicle and the lumen in the lamina are formed in the same vertebra. The lumen in the pedicle and the lumen in the lamina are formed in the same side of the vertebra relative to the spinous process. In some embodiments, at least a portion of the lumen in the lamina has a curved or non-linear configuration. In some embodiments, at least a portion of the lumen in the lamina has a straight or linear configuration. In some embodiments, the lumen in the lamina has a straight or linear configuration. The lumen in the lamina can be near the pars fracture. The lumen in the lamina can be adjacent to the pars fracture. The lumen in the lamina can be spaced apart from the pars fracture. The lumen in the lamina can be a distance from the pars fracture of 5 mm, 10 mm, 15 mm, 20 mm, more than 5 mm, more than 10 mm, more than 15 mm, more than 20 mm, less than 5 mm, less than 10 mm, less than 15 mm, less than 20 mm, or any range of two of the foregoing values. The lumen in the pedicle can be located on a first side of the pars fracture. The lumen in the pedicle can be located on the edge of the pedicle. The lumen in the pedicle can extend to or through the pars fracture. The lumen in the pedicle can be spaced apart from the pars fracture. The lumen in the lamina can be located on a second side of the pars fracture. The pars fracture can be between the lumen in the pedicle and the lumen in the lamina. The lumen in the lamina can be located on the edge of the lamina. The lumen in the lamina can extend to or through the pars fracture. The lumen in the lamina can be spaced apart from the pars fracture.

In some embodiments, the lumen in the lamina can connect to the lumen in the pedicle. The lumen in the lamina can intersect the lumen in the pedicle. The lumen in the lamina can extend into the lumen in the pedicle. The lumen in the lamina and the lumen in the pedicle can be aligned. The lumen in the lamina and the lumen in the pedicle can be coaxial. The lumen in the lamina and the lumen in the pedicle can be along a linear axis. The lumen in the lamina and the lumen in the pedicle can be along a straight pathway. The lumen in the lamina and the lumen in the pedicle can be along a linear trajectory. The lumen in the lamina and the lumen in the pedicle can be formed utilizing a straight drill. The lumen in the lamina and the lumen in the pedicle can be formed utilizing a single linear trajectory. In some embodiments, the lumen in the lamina has a straight or linear configuration and the lumen in the pedicle has a straight or linear configuration. The lumen in the pedicle and the lumen in the lamina can cross. In some embodiments, a single straight drill can be used to drill the lumen in the pedicle and the lumen in the lamina. The single straight drill can form the lumen in the pedicle and the lumen in the lamina along a straight trajectory. The single straight drill can form both lumens, the lumen in the pedicle and the lumen in the lamina. The drill can approach from a contralateral position relative to the pars fracture. The drill can approach from an ipsilateral position relative to the pars fracture. The path of the lumen in the pedicle and the path of the lumen in the lamina can extend from the edge of the pedicle to the edge of the lamina. The drill can approach along a trajectory that intersects the pedicle and the lamina. The drill can approach along a trajectory that intersects the pars fracture. The location of the lumen in the pedicle and the lumen in the lamina can be dependent on the individual anatomy of the patient, with examples provided in the reference drawings of the location of each.

In some embodiments, the lumen in the lamina has a curved configuration and the lumen in the pedicle has a curved configuration. The lumen in the lamina and the lumen in the pedicle can be continuous. The lumen in the lamina and the lumen in the pedicle can be along the same arc. The lumen in the lamina and the lumen in the pedicle can have the same radius of curvature. The lumen in the lamina and the lumen in the pedicle can be along a curved trajectory. The lumen in the lamina and the lumen in the pedicle can be formed utilizing a curved drill. The lumen in the lamina and the lumen in the pedicle can be formed utilizing a single curved trajectory. In some embodiments, the lumen in the lamina has a linear or non-linear configuration and the lumen in the pedicle has a linear or non-linear configuration. The lumen in the lamina and the lumen in the pedicle can have the same configuration. The lumen in the lamina and the lumen in the pedicle can have different configurations. In some embodiments, the lumen in the lamina does not intersect the lumen in the pedicle. The lumen in the lamina and the lumen in the pedicle can be separately formed. The lumen in the lamina and the lumen in the pedicle can be separated by a bony portion. The lumen in the lamina and the lumen in the pedicle can be separated by the pars fracture. The lumen in the pedicle can extend to the vertebral foramen and the lumen in the lamina can extend to the vertebral foramen. The lumen in the pedicle can extend through the pedicle. The lumen in the lamina can extend through the lamina. The lumen in the pedicle and the lumen in the lamina can be parallel or skewed relative to each other. The lumen in the pedicle and the lumen in the lamina can be non-crossing.

In some embodiments, the lumen in the lamina extends through the pars fracture. In some embodiments, the lumen in the pedicle extends through the pars fracture. The lumen in the lamina and the lumen in the pedicle can be along a trajectory through the pars fracture. The drill bit can extend through the pars fracture to form the lumen in the lamina. The drill bit can extend through the pars fracture to form the lumen in the pedicle. The drill bit can extend in a continuous path through the pars fracture. In some embodiments, the lumen in the lamina does not extend through the pars fracture. In some embodiments, the lumen in the pedicle does not extend through the pars fracture. The lumen in the lamina and the lumen in the pedicle can be along a trajectory separated from the pars fracture. The lumen in the lamina and the lumen in the pedicle can be along a trajectory that extends around the pars fracture. The lumen in the lamina can extend parallel to the pars fracture. The lumen in the pedicle can extend parallel to the pars fracture. The lumen in the lamina can be spaced apart from the pars fracture. The lumen in the pedicle can be spaced apart from the pars fracture.

In some embodiments, the lumen is formed in the pedicle and the lumen is formed in the lamina separately. In some embodiments, the lumen is formed in the pedicle first and the lumen is formed in the lamina second. In some embodiments, the lumen is formed in the lamina first and the lumen is formed in the pedicle second. In some embodiments, the lumen is formed in the pedicle and the lumen is formed in the lamina simultaneously. In some embodiments, the lumen is formed in the pedicle and the lumen is formed in the lamina in a sequential order. In some embodiments, the lumen is formed in the pedicle and the lumen is formed in the lamina in a continuous motion. In some embodiments, the lumen is formed in the pedicle and the lumen is formed in the lamina with a single drill. In some embodiments, the lumen is formed in the pedicle and the lumen is formed in the lamina with a single trajectory. In some embodiments, the lumen is formed in the pedicle and the lumen is formed in the lamina along a continuous pathway. In some embodiments, the lumen is formed in the pedicle and the lumen is formed in the lamina along a straight line. In some embodiments, the lumen is formed in the pedicle and the lumen is formed in the lamina along an arc.

In some embodiments, a portion of the pedicle and/or the lamina can be prepared for receiving bone tie 100. In some embodiments, a portion of the surface of the pedicle or lamina can be ground, scored, roughened, or sanded, such that the surface of the pedicle or lamina can better receive the bone tie 100. In some embodiments, the surgical procedure can include preparing the area near and/or around the pedicle or lamina by, for example, removing all or a portion of ligaments, cartilage, and/or other tissue.

In some embodiments, the pars interarticularis is prepared prior to securing the bone tie 100. In some embodiments, the area near and/or around a pars fracture can be prepared by removing all or a portion of ligaments, cartilage, and/or other tissue. In some embodiments, the granulation tissue can be removed and the bone on either side of the fracture can be exposed. The edges of bone fragments can be united. The bone fragments can be placed in proper position relative to each other. The bone fragments may be too far apart to fuse naturally. In some embodiments, graft material such as bone chips from adjacent bone can be placed into the pars fracture to promote healing. In some embodiments, graft material is placed over the pars fracture. In some embodiments, blood vessels that have been disrupted are repaired or cauterized.

The bone tie 100 can be advanced through the lumen in the pedicle of the vertebra. The bone tie 100 can be advanced through the lumen in the lamina of the vertebra. The bone tie 100 can be advanced by a tool configured to advance the head 136 through the lumens. In some embodiments, the bone tie 100 can have a bend or curve to facilitate directing the head 136 of the bone tie 100 through the lumen in the pedicle. In some embodiments, the bone tie 100 can have a straight or linear configuration to facilitate directing the head 136 of the bone tie 100 through the lumen in the pedicle. In some embodiments, the bone tie 100 can have a bend or curve to facilitate directing the head 136 of the bone tie 100 through the lumen in the lamina. In some embodiments, the bone tie 100 can have a straight or linear configuration to facilitate directing the head 136 of the bone tie 100 through the lumen in the lamina. The bone tie 100 can have any shape that allows the passage of the head 136 through the lumens.

During advancement, the head 136 of the bone tie 100 can be monitored under radiographic visualization. The head 136 can include one or more markers 144. In the illustrated embodiment, the head 136 can include the bore 146 configured to receive the marker 144. The marker 144 can be radiopaque. The marker 144 can facilitate placement of the head 136 through the lumen in the pedicle. The marker 144 can facilitate placement of the head 136 through the lumen in the lamina. The marker 144 can facilitate placement of the head 136 relative to a tool configured to retrieve the head 136. In some embodiments, the bone tie 100 can be advanced from the lumen in the pedicle to the lumen in the lamina. In some embodiments, the bone tie 100 can be advanced through intersecting lumens. In some embodiments, the bone tie 100 can be advanced through continuous lumens. In some embodiments, the bone tie 100 can be advanced directly from the lumen in the pedicle into the intersecting lumen in the lamina. In some embodiments, the bone tie 100 can be advanced from the lumen in the pedicle to the vertebral foramen. The bone tie 100 can be advanced from the vertebral foramen to the lumen in the lamina. The bone tie 100 can be advanced in any series of steps. In some embodiments, the bone tie 100 can be advanced from the lumen in the lamina to the lumen in the pedicle. In some embodiments, the bone tie 100 can be advanced directly from the lumen in the lamina into the intersecting lumen in the pedicle. In some embodiments, the bone tie 100 can be advanced from the lumen in the lamina to the vertebral foramen. The bone tie 100 can be advanced from the vertebral foramen to the lumen in the pedicle. In some embodiments, a tool configured to retrieve the head 136 can be utilized. The tool can be pulled to retract the head 136 from the lumens.

The bone tie 100 can be passed through the lumen in the pedicle and the lumen of the lamina of the vertebra. The bone tie 100 can form an arc from the pedicle to the lamina. In some embodiments, the bone tie 100 can form an arc from the pedicle to the lamina within the body of the vertebra. In some embodiments, the bone tie 100 can form an arc from the pedicle to the lamina through lumens that are connected. In some embodiments, the bone tie 100 can form an arc from the pedicle to the vertebral foramen to the lamina.

In some embodiments, the bone tie 100 is configured to form a loop around the pars fracture. The bone tie 100 forms a loop between the lamina and the pedicle. The bone tie 100 is disposed on the dorsal side of the pars fracture. The bone tie 100 is disposed on the ventral side of the pars fracture. In some embodiments, the bone tie 100 is configured to form a loop around the pars fracture on one side of the spine such as the right side. The bone tie 100 is configured to bring the fragments of the pars fracture together. In some embodiments, the bone tie 100 is configured to form a u-shaped configuration until the bone tie 100 is secured. The u-shaped configuration can extend underneath the pars fracture. In some embodiments, a portion of the bone tie 100 is underneath or on the ventral side of the pars fracture while the head 136 and the fastener section 106 can be above or on the dorsal side of the pars fracture. In some embodiments, the bone tie 100 only extends under and over the pars fracture. In some embodiments, the bone tie 100 does not extend through the pars fracture.

In some embodiments, the bone tie 100 extends through the pars fracture. The lumen in the pedicle can extend through the pars fracture. The lumen in the lamina can extend through the pars fracture. The bone tie 100 forms a loop between the lamina and the pedicle through the pars fracture. In some embodiments, the bone tie 100 is configured to form a loop through the pars fracture on one side of the spine such as the right side. The bone tie 100 is configured to bring the fragments of the pars fracture together. In some embodiments, the bone tie 100 is configured to form a u-shaped configuration until the bone tie 100 is secured. The u-shaped configuration can extend through the pars fracture. In some embodiments, a portion of the bone tie 100 is through the pars fracture while the head 136 and the fastener section 106 can be above or on the dorsal side of the pars fracture. In some embodiments, the bone tie 100 only extends through and over the pars fracture. In some embodiments, the bone tie 100 does not extend underneath or on the dorsal side of the pars fracture.

In some embodiments, the head section 116 can be removed after the bone tie 100 is positioned underneath the pars fracture. The bone tie 100 can be cut or severed near the neck section 114. The bone tie 100 can be cut or severed to remove the head section 116 and a portion of the neck section 114. The head section 116 can be discarded.

The bone tie 100 can be secured. The bone tie 100 can be wrapped around the outside, dorsal portion of the vertebral body. The bone tie 100 can be fastened to form a loop. The neck section 114 can be advanced through the lumen 118 of the fastener section 106. While the neck section 114 is being advanced, the ratchet 122 can extend into the groove 132. The third section 112 can be advanced through the lumen 118 of the fastener section 106. While the third section 112 is being advanced, the ratchet 122 can extend into the groove 130. The second section 110 can be advanced through the lumen 118 of the fastener section 106. While the second section 110 is being advanced, the ratchet 122 can extend into the groove 126. The ratchet 122 can engage the one or more gears 128. The ratchet 122 can allow the second section 110 to travel through the lumen 118 of the fastener section 106 in one direction, but limit or prevent travel in the opposite direction.

The bone tie 100 can be tightened in one direction. The bone tie 100 can be tightened by advancing a portion of the bone tie 100 through the fastener section 106. The fastener section 106 can be tightened along the length of the bone tie 100. In some embodiments, the neck section 114, the third section 112, and at least a portion of the second section 100 can be advanced through the fastener section 106 such that the area disposed within the loop formed by the bone tie 100 is reduced. The fastener section 106 can engage the one or more gears 128 of the second section 110. The one or more gears 128 can be shaped to allow each gear 128 to displace the ratchet 122 of fastener section 106 in only one direction. The bone tie 100 can be tightened until flush with the surface of the vertebra. In some embodiments, the fastener section 106 can be positioned near the lumen in the lamina. In some embodiments, the fastener section 106 can be positioned near the lumen in the pedicle. The bone tie 100 can be tightened until the fragments of the pars fracture are moved into the desired position. The bone tie 100 can be tightened until further tightening is not possible. The bone tie 100 can be tightened to provide the desired compressive force. The bone tie 100 can be tightened to reduce the gap between bone fragments.

The bone tie 100 can be tightened to exert a force on the pars fracture. The bone tie 100 can be tightened to exert a stabilizing force on the bone fragments. The bone tie 100 can be tightened to position edges of the fracture into abutment to promote healing. The bone tie 100 can apply a compressive force to the bone fragments to promote healing. The bone tie 100 can promote fusion. The bone tie 100 can position the bone to promote new bone formation. The bone tie 100 can apply a force to realign bone fragments. The bone tie 100 can apply a force to correct spondylolysis. The bone tie 100 can apply a force to correct spondylolisthesis. The bone tie 100 can apply a force to prevent sliding of the vertebra.

In some embodiments, the bone tie 100 remains in place to correct the pars fracture. In some embodiments, the bone tie 100 remains in place for one week, two weeks, three weeks, four weeks, five weeks, six weeks, one month, two months, three months, four months, five months, six months, one year, two years, or any range of two of the foregoing values. In some embodiments, the bone tie 100 is removed after the pars fracture is corrected. In some embodiments, the bone tie 100 is removed after one week, two weeks, three weeks, four weeks, five weeks, six weeks, one month, two months, three months, four months, five months, six months, one year, or any range of two of the foregoing values. In some embodiments, the bone tie 100 is removed and replaced. In some embodiments, the bone tie 100 is replaced with another bone tie that exerts a stronger compressive force. In some embodiments, the bone tie 100 may comprise a bioabsorbable or bioresorbable material.

In some embodiments, the bone tie 100 can be configured to stabilize the pars fracture by securing the pedicle of the vertebra to the lamina of the vertebra. The bone tie 100 can be placed into a suitable position relative to the vertebra. The bone tie 100 can be placed into a suitable position that allows a distal portion of the bone tie 100 to be inserted into the lumen 118 of the fastener section 106. In some embodiments, the fastener section 106 is positioned near one of the lumens once the bone tie 100 is tightened. In some embodiments, the fastener section 106 is positioned near the lamina once the bone tie 100 is tightened. In some embodiments, the fastener section 106 is positioned near the pedicle once the bone tie 100 is tightened. The bone tie 100 can be configured to substantially encircle at least a portion of the pars fracture. In some embodiments, the bone tie 100 forms a loop about the pedicle and the lamina of the same vertebra. In some embodiments, the bone tie 100 forms a loop about the pars fracture.

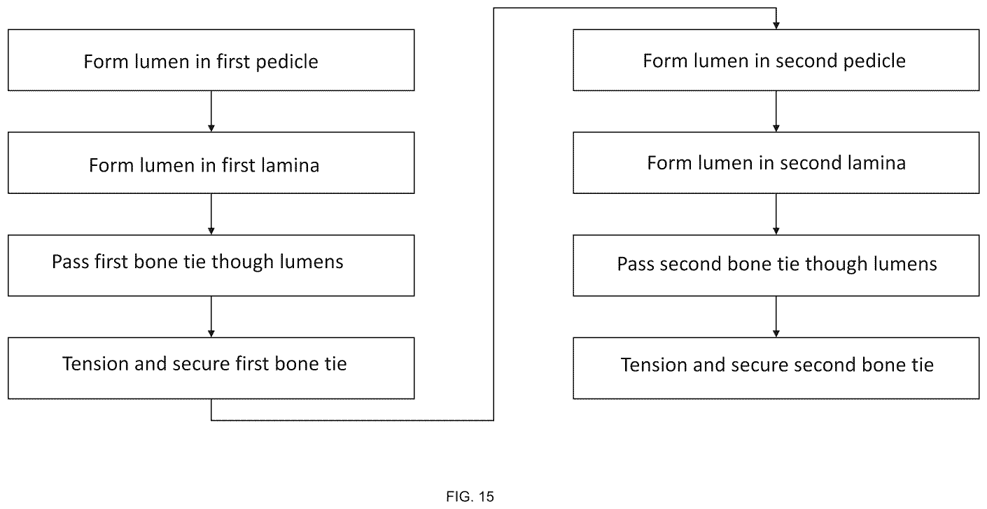

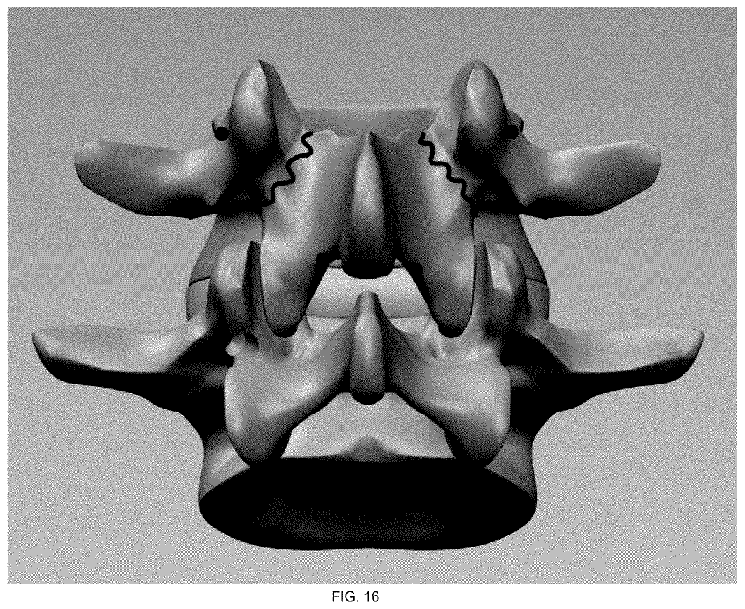

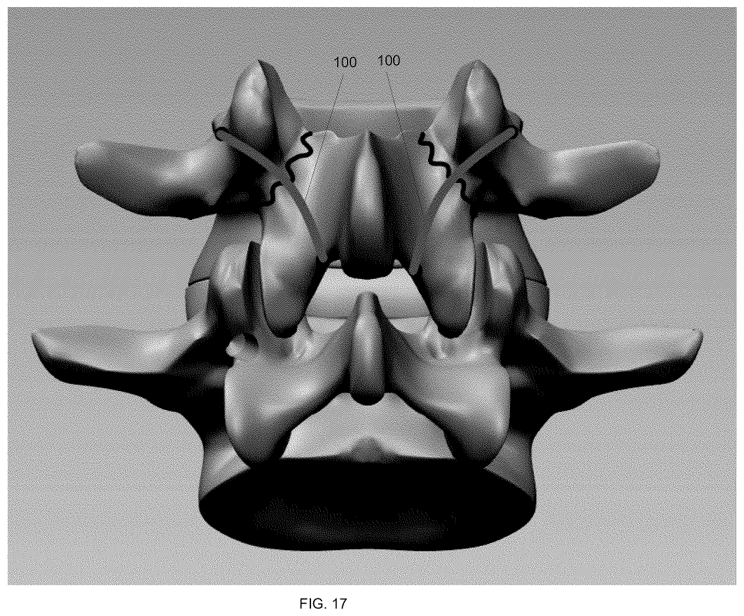

FIG. 15 is a flow chart for a method of using the bone tie 100. FIG. 16 illustrates a view of a vertebra with two pars fractures. FIG. 17 is a view of the bone tie 100 positioned to repair the pars fractures. The methods of use can include any steps described herein.

The bone ties 100 can be utilized to fuse bone fragments of a single vertebra. The first bone tie 100 can be used to fuse the first pedicle to the first lamina of the vertebra. The second bone tie 100 can be used to fuse the second pedicle to the second lamina of the vertebra. The first bone tie 100 can be positioned on the right side of the patient. The second bone tie 100 can be positioned on the left side of the patient. In some embodiments, each pars fracture is repaired by a single bone tie 100. The first bone tie 100 forms a loop around the first pars fracture. The second bone tie 100 forms a loop around the second pars fracture. In some embodiments, one bone tie 100 can be used to stabilize a fracture on the right side of the spinous process and another bone tie 100 can be used to stabilize a fracture on the left side of the spinous process.

In some embodiments, a pathway is formed in the first pedicle. In some embodiments, the lumen can extend entirely through the first pedicle. In other embodiments, the lumen can extend partially through the first pedicle. In some embodiments, at least a portion of the lumen in the first pedicle has a curved or non-linear configuration. In some embodiments, at least a portion of the lumen in the first pedicle has a straight or linear configuration. In some embodiments, the lumen in the first pedicle has a straight or linear configuration. In some embodiments, the lumen in the first pedicle has a curved or non-linear configuration.

In some embodiments, a pathway is formed in the first lamina. In some embodiments, the lumen can extend entirely through the first lamina. In other embodiments, the lumen can extend partially through the first lamina. In some embodiments, at least a portion of the lumen in the first lamina has a curved or non-linear configuration. In some embodiments, at least a portion of the lumen in the first lamina has a straight or linear configuration. In some embodiments, the lumen in the first lamina has a straight or linear configuration. In some embodiments, the lumen in the first lamina has a curved or non-linear configuration.

In some embodiments, the lumen in the first lamina can connect to the lumen in the first pedicle. The lumen in the first lamina and the lumen in the first pedicle can be along a straight trajectory. In other embodiments, the lumen in the first lamina does not intersect the lumen in the first pedicle. The lumen in the first pedicle can extend to the vertebral foramen and the lumen in the first lamina can extend to the vertebral foramen. In some embodiments, the lumen is formed in the first pedicle and the lumen is formed in the first lamina separately. In some embodiments, the lumen is formed in the first pedicle and the lumen is formed in the first lamina simultaneously.

In some embodiments, a portion of the first pedicle and/or the first lamina can be prepared for receiving first bone tie 100. In some embodiments, the first pars interarticularis is prepared prior to securing the first bone tie 100. The edges of bone fragments can be united. In some embodiments, a forward slip or slide of the vertebra is corrected. The vertebra is brought into alignment with other vertebra of the spine. In some embodiments, the vertebra has slipped forward and the ends of the bone fragments are not near each other. The method can include correcting the slipped vertebra.

The first bone tie 100 can be advanced through the lumen in the first pedicle of the vertebra. The first bone tie 100 can be advanced through the lumen in the first lamina of the vertebra. During advancement, the head 136 of the first bone tie 100 can be monitored under radiographic visualization. The first bone tie 100 can be advanced from the lumen in the first pedicle to the lumen in the first lamina. The first bone tie 100 can be advanced from the lumen in the first lamina to the lumen in the first pedicle. In some embodiments, the first bone tie 100 can be advanced through intersecting lumens. In some embodiments, the first bone tie 100 can be advanced along a straight trajectory between the lumen in the first lamina and the lumen in the first pedicle. In some embodiments, the first bone tie 100 can be advanced from the lumen in the first pedicle to the vertebral foramen. The first bone tie 100 can be advanced from the vertebral foramen to the lumen in the first lamina. In some embodiments, the first bone tie 100 can be advanced along a curved trajectory between the lumen in the first lamina and the lumen in the first pedicle. The first bone tie 100 can form an arc from the first pedicle to the first lamina.

The first bone tie 100 is configured to form a loop around the first pars fracture. In some embodiments, the first bone tie 100 is configured to form a loop around the first pars fracture on one side of the spine such as the right side. The first bone tie 100 is configured to bring the fragments of the first pars fracture together. In some embodiments, the first bone tie 100 can extend underneath the first pars fracture. In some embodiments, a portion of the first bone tie 100 is underneath the first pars fracture while the head 136 and the fastener section 106 can be above the first pars fracture. In some embodiments, the head section 116 can be removed after the first bone tie 100 is positioned underneath the first pars fracture. In some embodiments, the first bone tie 100 does not extend through the pars fracture. The lumen in the first lamina and the lumen in the first pedicle does not extend through the pars fracture. In some embodiments, the bone tie 100 extends through the pars fracture. The lumen in the first lamina and the lumen in the first pedicle extend through the pars fracture.

The first bone tie 100 can be secured. The first bone tie 100 can be wrapped around the outside, dorsal portion of the vertebra. The first bone tie 100 can be fastened to form a loop. The first bone tie 100 can be tightened in one direction. The first bone tie 100 can be tightened until the fragments of the first pars fracture are stabilized in the desired position. The first bone tie 100 can be tightened to exert a force on the first pars fracture. In some embodiments, the first bone tie 100 can be configured to stabilize the first pars fracture by securing the first pedicle of the vertebra to the first lamina of the vertebra. In some embodiments, the first bone tie 100 forms a loop about the first pedicle and the first lamina of the same vertebra.

The second bone tie 100 can be used to fuse the second pedicle to the second lamina of the vertebra. The second bone tie 100 can be positioned on the left side of the patient. In some embodiments, the second bone tie 100 forms a loop around the second pars fracture.

In some embodiments, a pathway is formed in the second pedicle. In some embodiments, the lumen can extend entirely through the second pedicle. In other embodiments, the lumen can extend partially through the second pedicle. In some embodiments, at least a portion of the lumen in the second pedicle has a curved or non-linear configuration. In some embodiments, at least a portion of the lumen in the second pedicle has a straight or linear configuration. In some embodiments, the lumen in the second pedicle has a straight or linear configuration. In some embodiments, the lumen in the second pedicle has a curved or non-linear configuration.

In some embodiments, a pathway is formed in the second lamina. In some embodiments, the lumen can extend entirely through the second lamina. In other embodiments, the lumen can extend partially through the second lamina. In some embodiments, at least a portion of the lumen in the second lamina has a curved or non-linear configuration. In some embodiments, at least a portion of the lumen in the second lamina has a straight or linear configuration. In some embodiments, the lumen in the second lamina has a straight or linear configuration. In some embodiments, the lumen in the second lamina has a curved or non-linear configuration.

In some embodiments, the lumen in the second lamina can connect to the lumen in the second pedicle. The lumen in the second lamina and the lumen in the second pedicle can be along a straight trajectory. In other embodiments, the lumen in the second lamina does not intersect the lumen in the second pedicle. The lumen in the second pedicle can extend to the vertebral foramen and the lumen in the second lamina can extend to the vertebral foramen. In some embodiments, the lumen is formed in the second pedicle and the lumen is formed in the second lamina separately. In some embodiments, the lumen is formed in the second pedicle and the lumen is formed in the second lamina simultaneously.

In some embodiments, a portion of the second pedicle and/or the second lamina can be prepared for receiving second bone tie 100. In some embodiments, the second pars interarticularis is prepared prior to securing the second bone tie 100. The edges of bone fragments can be united. In some embodiments, a forward slip or slide of the vertebra is corrected. The vertebra is brought into alignment with other vertebra of the spine. In some embodiments, the vertebra has slipped forward and the ends of the bone fragments are not near each other. The method can include correcting the slipped vertebra.

The second bone tie 100 can be advanced through the lumen in the second pedicle of the vertebra. The second bone tie 100 can be advanced through the lumen in the second lamina of the vertebra. During advancement, the head 136 of the second bone tie 100 can be monitored under radiographic visualization. The second bone tie 100 can be advanced from the lumen in the second pedicle to the lumen in the second lamina. The second bone tie 100 can be advanced from the lumen in the second lamina to the lumen in the second pedicle. In some embodiments, the second bone tie 100 can be advanced through intersecting lumens. In some embodiments, the second bone tie 100 can be advanced along a straight trajectory between the lumen in the second lamina and the lumen in the second pedicle. In some embodiments, the second bone tie 100 can be advanced from the lumen in the second pedicle to the vertebral foramen. The second bone tie 100 can be advanced from the vertebral foramen to the lumen in the second lamina. In some embodiments, the second bone tie 100 can be advanced along a curved trajectory between the lumen in the second lamina and the lumen in the second pedicle. The second bone tie 100 can form an arc from the second pedicle to the second lamina.

In some embodiments, the second bone tie 100 is configured to form a loop around the second pars fracture. In some embodiments, the second bone tie 100 is configured to form a loop around the second pars fracture on one side of the spine such as the left side. The second bone tie 100 is configured to bring the fragments of the second pars fracture together. In some embodiments, the second bone tie 100 can extend underneath the second pars fracture. In some embodiments, a portion of the second bone tie 100 is underneath the second pars fracture while the head 136 and the fastener section 106 can be above the second pars fracture. In some embodiments, the head section 116 can be removed after the second bone tie 100 is positioned underneath the second pars fracture. In some embodiments, the second bone tie 100 does not extend through the pars fracture. The lumen in the second lamina and the lumen in the second pedicle does not extend through the pars fracture. In some embodiments, the bone tie 100 extends through the pars fracture. The lumen in the second lamina and the lumen in the second pedicle extend through the pars fracture.

The second bone tie 100 can be secured. The second bone tie 100 can be wrapped around the outside, dorsal portion of the vertebra. The second bone tie 100 can be fastened to form a loop. The second bone tie 100 can be tightened in one direction. The second bone tie 100 can be tightened until the fragments of the second pars fracture are stabilized in the desired position. The second bone tie 100 can be tightened to exert a force on the second pars fracture. In some embodiments, the second bone tie 100 can be configured to stabilize the second pars fracture by securing the second pedicle of the vertebra to the second lamina of the vertebra. In some embodiments, the second bone tie 100 can from a loop about the second pedicle and the second lamina of the same vertebra.

The steps of the method can be performed in any order. In some embodiments, the lumen in the first pedicle is drilled before the lumen in the first lamina. In some embodiments, the lumen in the first pedicle is drilled before the lumen in the second pedicle. In some embodiments, the lumen in the first lamina is drilled before lumen in the second lamina. In some embodiments, the first bone tie 100 is advanced through the lumen in the first pedicle and the lumen in the first lamina before the second bone tie 100 is advanced through the lumen in the second pedicle and the lumen in the second lamina. In some embodiments, the first bone tie 100 is secured before the second bone tie is advanced through the lumen in the second pedicle and the lumen in the second lamina. In some embodiments, the first bone tie 100 is tightened before the second bone tie 100 is advanced through the lumen in the second pedicle and the lumen in the second lamina. In some embodiments, the first bone tie 100 is secured before the second bone tie 100 is secured. In some embodiments, the first bone tie 100 is tightened before the second bone tie 100 is tightened. In some embodiments, the first bone tie 100 is tightened before the lumen in the second pedicle is drilled. In some embodiments, the first bone tie 100 is tightened before the lumen in the second lamina is drilled. In some embodiments, the first bone tie 100 is tightened after the lumen in the second pedicle is drilled. In some embodiments, the first bone tie 100 is tightened after the lumen in the second lamina is drilled. In some embodiments, the first bone tie 100 is tightened after the second bone tie is advanced through the lumen in the second pedicle and the lumen in the second lamina.

The bone tie 100 can have any shape. The bone tie 100 can have a shape to conform to a portion of the bone. In some embodiments, the fastener section 106 can be configured to remain within the body of the patient. The fastener section 106 can be near the proximal end 102. The fastener section 106 can have a shape configured to conform to the shape of the vertebra. The fastener section 106 can have a flat surface configured to engage the vertebra. The ratchet 122 can be disposed within a lumen in a portion of the bone. The lumen can prevent encroachment of tissue relative to the ratchet 122. The fastener section 106 can have an enlarged head configured to distribute forces to the vertebra. The fastener section 106 can have an enlarged head configured to prevent subsidence into the vertebra.

In some embodiments, the first section 108 can be configured to remain within the body of the patient. The first section 108 can be closer to the proximal end 102 than the distal end 104. The first section 108 can have a first cross-sectional shape configured to conform to the shape of one or more of the lumens. The first section 108 can be rounded. The rounded surface or edges may facilitate engagement with a rounded lumen or pathway.

In some embodiments, the second section 110 can be configured to remain within the body of the patient. In some embodiments, a portion of the second section 110 can be configured to remain within the body of the patient. In some embodiments, a portion of the second section 110 is cut after tightening to remove excess length of the bone tie 100. The second section 110 can be closer to the proximal end 102 than the distal end 104. The second section 110 can have a first cross-sectional shape configured to conform to the shape of the bone. The second section 110 can be rounded. The rounded surface or edges may facilitate engagement with a rounded lumen or pathway.

In some embodiments, the third section 112, or a portion thereof, can be configured to remain within the body of the patient. In some embodiments, the third section 112 is cut after tightening to remove excess length of the bone tie 100. The third section 112 can have raised edges relative to the groove 130. The raised edges can slide along corresponding groves in the lumen 118 of the fastener section 106. The raised edges of the second section 110 and the third section 112 can be continuous.

The first section 108, the second section 110, and the third section 112 can be any portion of the length of the bone tie 100. In some embodiments, the second section 110 is at least 30% of the length of the bone tie 100. In some embodiments, the third section 112 is at least 30% of the length of the bone tie 100. Other configurations are contemplated. The first section 108 can be 5% of the total length, 10% of the total length, 15% of the total length, 20% of the total length, 25% of the total length, 30% of the total length, 35% of the total length, 40% of the total length, 45% of the total length, 50% of the total length, or any range of the foregoing values. The second section 110 can be 5% of the total length, 10% of the total length, 15% of the total length, 20% of the total length, 25% of the total length, 30% of the total length, 35% of the total length, 40% of the total length, 45% of the total length, 50% of the total length, 55% of the total length, 60% of the total length, 65% of the total length, 70% of the total length, 75% of the total length, 80% of the total length, 85% of the total length, 90% of the total length, 95% of the total length, or any range of the foregoing values. The third section 112 can be 5% of the total length, 10% of the total length, 15% of the total length, 20% of the total length, 25% of the total length, 30% of the total length, 35% of the total length, 40% of the total length, 45% of the total length, 50% of the total length, or any range of the foregoing values. In some embodiments, the length of second section 110 can be about equal to the length of the third section 112. In some embodiments, the length of second section 110 can be greater than the length of the third section 112. In some embodiments, the length of second section 110 can be less than the length of the third section 112.

The bone tie 100 can be configured to loop around at least a portion of the anatomy. In some embodiments, the bone tie 100 completely encircles the anatomy. In some embodiments, the bone tie 100 completely encircles the pars fracture. In some embodiments, the bone tie 100 completely encircles a portion of the vertebral body.

The first section 108 can have a uniform shape. The first section 108 can have a substantially cuboidal shape or a substantially cylindrical shape. The second section 110 can have a uniform shape. The second section 110 can have a substantially cuboidal shape or a substantially cylindrical shape. The third section 112 can have a uniform shape. The third section 112 can have a substantially cuboidal shape or a substantially cylindrical shape. The first section 108 and the third section 112 can have the same or similar shape.

The bone tie 100 can be utilized alone. The bone tie 100 can be utilized in connection with another bone tie 100. The bone tie 100 can be utilized in connection with an implant. The bone tie 100 can be utilized in connection with an interbody implant. The bone tie 100 can be utilized in connection with a facet implant. The bone tie 100 can be utilized in connection with fusion material. The bone tie 100 can be utilized in connection with bone grafts. The bone tie 100 can be utilized in connection with any substance. The bone tie 100 can be utilized in connection with any biologic and/or chemical substance, including, but not limited to, medicine, adhesives, etc., and/or a bone graft, including, but not limited to, autograft, allograft, xenograft, alloplastic graft, a synthetic graft, and/or combinations of grafts, medicines, and/or adhesives. In some embodiments, a bone graft can be used to support the area of the pars fracture. While exemplary references are made with respect to vertebra, in some embodiments another bone can be involved. While specific reference may be made to a specific vertebra and/or subset and/or grouping of vertebrae, it is understood that any vertebra and/or subset and/or grouping, or combination of vertebrae can be used. The bone tie 100 can deliver a substance. The pars fracture can be packed with a substance. The lumen in the pedicle can be packed with a substance. The lumen in the lamina can be packed with a substance. The bone tie 100 can be configured to retain, carry and/or otherwise deliver a substance to aid in fusion, such as, for example, medicines, adhesives, bone graft, and/or combinations of substances.

The bone tie 100 can have several advantages. The bone tie 100 can allow for simplified subsequent removal techniques versus traditional hardware. The bone tie 100 can be easily cut to be removed. The bone tie 100 can be removed after fusion. The bone tie 100 can be adjusted during a procedure to adjust the tension on the pars fracture. The bone tie 100 can be adjusted during a procedure to increase the tension on the pars fracture. The bone tie 100 can be adjusted during a procedure to increase the compression on bone fragments. The bone tie 100 can be removed during a procedure and a new bone tie 100 can be positioned in order to modify the tension on the pars fracture. The bone tie 100 can be removed during a procedure to decrease the tension on the pars fracture. The bone tie 100 can removed and replaced with another bone tie 100. In some embodiments, the bone tie 100 can absorb over time within the body of the patient. The bone tie 100 can be advantageously tightened in one direction. The bone tie 100 can maintain the tension under normal anatomical loads.

The bone tie 100 can be utilized to stabilize bone fragments of a pars fracture. The bone tie 100 can be utilized to position the bone fragments to promote fusion. The bone tie 100 can be utilized to correct or improve the misalignment or slip of a vertebra. The bone tie 100 can be utilized to allow the stress fracture to heal. The bone tie 100 can wrap around the pars fracture. In some embodiments, the bone tie 100 does not extend through the pars interarticularis. The bone tie 100 can be utilized to bridge the two sides of the fracture. The bone tie 100 can be utilized to allow for healing of the fracture. The bone tie 100 can be utilized to allow for stabilization of the fracture. The bone tie 100 can be utilized to reconstruct the posterior bony structure. The bone tie 100 can be utilized to preserve motion. The bone tie 100 can be utilized to apply compression to the pars fracture. The bone tie 100 can be utilized to increase compression. The bone tie 100 can be utilized to reduce the gap in the pars fracture to enhance the fusion rate. The bone tie 100 can be utilized to restore the stability of the spine.

In some embodiments described herein, the bone tie 100 can be used to stabilize and/or fixate a single vertebra. The bone tie 100 wraps around the pars fracture from the pedicle to the lamina. The bone tie 100 can be configured to reduce pain associated with a pars fracture. The bone tie 100 can be configured to reduce further degradation of a spine. The bone tie 100 can be configured to reduce further degradation of a lumbar vertebra. The bone tie 100 can be configured to reduce movement of bone fragments until the fragments of the pars interarticularis have fused. The bone tie 100 can be configured to stabilize the pars interarticularis by securing the pedicle of the vertebra to the lamina of the vertebra.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. For all the embodiments described above, the steps of the methods need not be performed sequentially. Thus, it is intended that the scope of the present invention herein disclosed should not be limited by the particular disclosed embodiments described above, but should be determined only by a fair reading of the claims that follow.

What is claimed is:

1. A method of treating a patient is provided, the method comprising:
forming a first lumen in a pedicle on a first side of a pars fracture;
forming a second lumen in a lamina on a second side of the pars fracture;
positioning a bone tie through the first lumen and the second lumen, the bone tie comprising a distal end and a fastener section; and
tightening the bone tie by passing the distal end of the bone tie through the fastener section of the bone tie.

2. The method of claim 1, wherein forming the first lumen comprises drilling a hole entirely through the pedicle.

3. The method of claim 1, wherein forming the first lumen comprises drilling a hole partially through the pedicle.

4. The method of claim 1, wherein forming the second lumen comprises drilling a hole entirely through the lamina.

5. The method of claim 1, wherein forming the second lumen comprises drilling a hole partially through the lamina.

6. The method of claim 1, wherein forming the second lumen comprises connecting the second lumen and the first lumen.

7. The method of claim 1, wherein positioning the bone tie comprises wrapping the bone tie around the outside portion of a vertebra.

8. The method of claim 1, wherein tightening the bone tie further comprises reducing a unilateral pars fracture.

9. The method of claim 1, wherein tightening the bone tie further comprises applying compression to the pars fracture.

10. The method of claim 1, wherein tightening the bone tie further comprises uniting fractured ends of the pars interarticularis.

11. The method of claim 1, further comprising removing the distal end of the bone tie.

12. The method of claim 1, further comprising removing the bone tie after the pars fracture heals.

13. The method of claim 1, wherein the bone tie comprises a bioabsorbable or bioresorbable material.

14. A method of treating a patient is provided, the method comprising:
forming a first lumen in a first pedicle on a first side of a first pars fracture;
forming a second lumen in a first lamina on a second side of the first pars fracture;
positioning a first bone tie through the first lumen and the second lumen, the first bone tie comprising a distal end and a fastener section;
tightening the first bone tie by passing the distal end of the first bone tie through the fastener section of the first bone tie;
forming a third lumen in a second pedicle on a first side of a second pars fracture;
forming a fourth lumen in a second lamina on a second side of the second pars fracture;
positioning a second bone tie through the third lumen and the fourth lumen, the second bone tie comprising a distal end and a fastener section; and
tightening the second bone tie by passing the distal end of the second bone tie through the fastener section of the second bone tie.

15. The method of claim 14, wherein forming the second lumen comprises connecting the second lumen and the first lumen.

16. The method of claim 14, wherein forming the fourth lumen comprises connecting the fourth lumen and the third lumen.

17. The method of claim 14, wherein tightening the first bone tie and tightening the second bone tie further comprises reducing a bilateral pars fracture.

18. The method of claim 14, wherein tightening the first bone tie further comprises applying compression to the first pars fracture and tightening the second bone tie comprises applying compression to the second pars fracture.

19. The method of claim 14, wherein tightening the first bone tie and tightening the second bone tie further comprises correcting a slip of a vertebra.

20. The method of claim 14, wherein tightening the first bone tie and tightening the second bone tie further comprises correcting a displaced fracture.
